(19) United States
(12) Patent Application Publication
(10) Pub. No.: US 2009/0264906 A1
(43) Pub. Date: Oct. 22, 2009

(54) Cuff Device

(75) Inventor: Paula McDonnell, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/107,501

(22) Filed: Apr. 22, 2008

(51) Int. Cl. A61B 17/08; A61M 29/00

(52) U.S. Cl. 606/151; 604/96.01

(57) ABSTRACT

A cuff device configured to treat a site of a lesion in a vessel includes a flexible material having a first surface and a second surface. The flexible material is configured to be wrapped around an exterior surface of the vessel so that the first surface of the flexible material is in contact with an exterior surface of the vessel. The cuff device includes an opening that extends from the first surface to the second surface. The opening is constructed and arranged to allow a needle of a syringe to pass therethrough.

### BACKGROUND OF THE INVENTION

1. Field of the Invention

The present invention is generally related to a device for treating a lesion in a vessel, particularly a chronic lesion in a vessel located in a lower limb.

2. Background of the Invention

In a typical percutaneous transluminal coronary angioplasty (PTCA) procedure, a guiding catheter is percutaneously introduced into the cardiovascular system of a patient. The guide catheter is advanced through a vessel until the distal end thereof is at the desired location in the vasculature. A guidewire and a dilatation catheter having a balloon on the distal end thereof are introduced into the guiding catheter with the guidewire sliding through the dilatation catheter. The guidewire is first advanced out of the guiding catheter into the patient's coronary vasculature, and the dilatation catheter is advanced over the previously advanced guidewire until the dilatation balloon is properly positioned across the lesion.

Once in position, the flexible, expandable, preformed balloon is inflated to a predetermined size with a liquid or gas at relatively high pressures (e.g., about ten to twelve atmospheres) to radially compress the arthrosclerotic plaque in the lesion against the inside of the vessel wall and thereby dilate the lumen of the vessel. The balloon is then deflated to a small profile so that the dilatation catheter may be withdrawn from the patient's vasculature and blood flow resumed through the dilated vessel.

Vessels which have undergone angioplasty frequently suffer from restenosis, which is an abrupt closure of the vessel that may quickly block the flow of blood. Therefore, therapeutic substances, such as anti-coagulants, are typically applied to an area of the vessel which has recently undergone angioplasty, usually immediately following the procedure.

Different devices have been used to deliver the therapeutic substances to a vessel that has recently undergone angioplasty. For example, porous balloon catheters have been used to deliver therapeutic substances from within the lumen of the vessel. Generally, such catheters are configured similar to conventional dilatation balloon catheters with the modification of small holes formed into the balloon through which a therapeutic substance may flow under pressure. The substance is also used as the inflation medium for the balloon. Although the inflated balloon places the perfusion outlets in close proximity to the vessel wall to be treated, expulsion of the therapeutic substance may require full inflation of the balloon, which may temporarily block the vessel being treated. Interruption of blood flow through the vessel is undesirable because it can cause pain and discomfort to the patient.

Another example of a device that is configured to deliver a therapeutic substance (or substances) to the area of the vessel that has recently undergone angioplasty is a prosthetic device having a matrix material that is placed on the outside of the vessel. The prosthetic device incorporates the therapeutic substance and locally eludes the substance at a predefined rate. The matrix material is configured to permit the loading of an adequate quantity of the therapeutic substance, and elude the therapeutic substance at an appropriate well-defined rate over time.

It is desirable to treat the area of the vessel that has recently undergone angioplasty with greater control in terms of the amount and timing of the therapeutic substance being provided to the area, while minimizing discomfort to the patient. In addition, it is desirable to be able to more directly treat the site of the lesion.

### SUMMARY OF THE INVENTION

Embodiments of the present invention describe an apparatus and method to treat a site of a lesion in a vessel. Embodiments described herein may be particularly suited for treating chronic lesions located in the lower limbs, such as the femoral artery, the peroneal artery, or the posterior artery.

According to an aspect of the present invention, there is provided a cuff device configured to treat a site of a lesion in a vessel. The cuff device includes a flexible material having a first surface and a second surface. The flexible material is configured to be wrapped around an exterior surface of the vessel so that the first surface of the flexible material is in contact with an exterior surface of the vessel. The cuff device also includes an opening that extends from the first surface to the second surface. The opening is constructed and arranged to allow a needle of a syringe to pass therethrough.

According to an aspect of the invention, there is provided a method for treating a site of a lesion in a vessel. The method also includes placing a flexible material around an exterior surface of the vessel, and locating an opening in the flexible material. The method includes passing a needle of a syringe through the opening and into the vessel, and injecting the vessel with a therapeutic substance with the syringe.

According to an aspect of the invention, there is provided a method for treating a site of a lesion in a vessel. The method includes placing a flexible material of a cuff device around an exterior surface of the vessel, and passing a tube of the cuff device that extends from the flexible material through skin. The method also includes passing a needle of a syringe through the tube and through an opening in the flexible material, and injecting the vessel with a therapeutic substance with the syringe.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 is a schematic diagram of a vessel with a lesion;

FIG. 2 is a schematic diagram of a balloon catheter being tracked to the site of the lesion of FIG. 1;

FIG. 3 is a schematic diagram of a balloon of the balloon catheter being expanded at the site of the lesion;

FIG. 4 is a schematic diagram of a cuff device according to an embodiment of the invention surrounding the vessel of FIG. 1 at the site of the lesion;

FIG. 5 is a schematic perspective view of the cuff device of FIG. 4;

FIG. 6 is a schematic top view of the cuff device of FIG. 5 in an unwrapped condition;

FIG. 7 is a schematic cross-sectional view of the cuff device of FIG. 5;

FIG. 8 is a schematic perspective view of a cuff device according to an embodiment of the invention;

FIG. 9 is a schematic top view of the cuff device of FIG. 8 in an unwrapped condition;

FIG. 10 is a schematic cross-sectional view of the cuff device of FIG. 8 with a cover; and

FIG. 11 is a schematic view of the cuff device of FIG. 10 surrounding the vessel at the site of the lesion.

### DETAILED DESCRIPTION OF EMBODIMENTS OF THE INVENTION

The following detailed description is merely exemplary in nature and is not intended to limit the invention or the application and use of the invention. Furthermore, there is no intention to be bound by any expressed or implied theory presented in the preceding technical field, background, brief summary or the following detailed description.

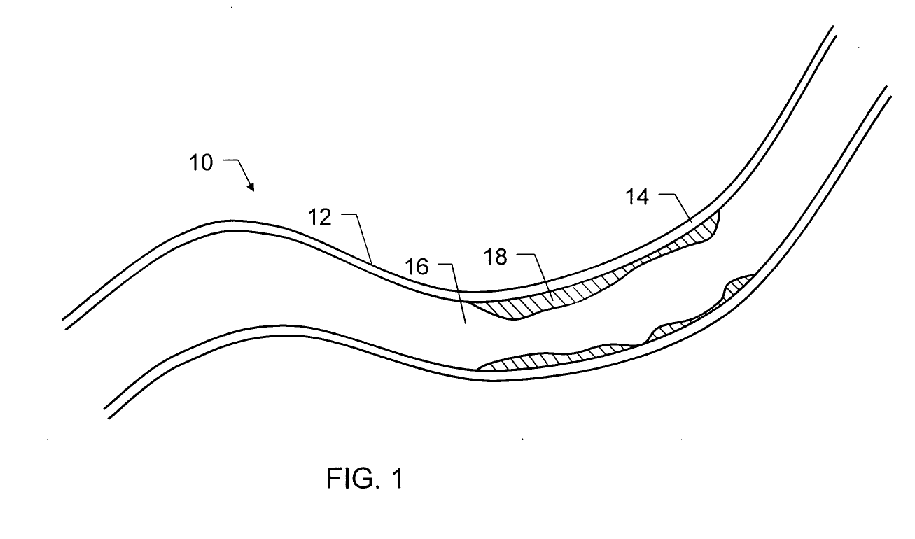

FIG. 1 illustrates a vessel 10 having an outer surface 12, and a wall 14 that defines a lumen 16. A lesion 18 is located at a site within the lumen 16. The lesion 18 may include atheromatous plaque that extends from the wall 14, and may substantially or even completely block flow of blood through the vessel 10. In an embodiment, the vessel 10 is a peripheral artery that is located in one of the lower limbs of a patient, and may be accessed surgically, such as for example the femoral artery, the peroneal artery, or the posterior artery.

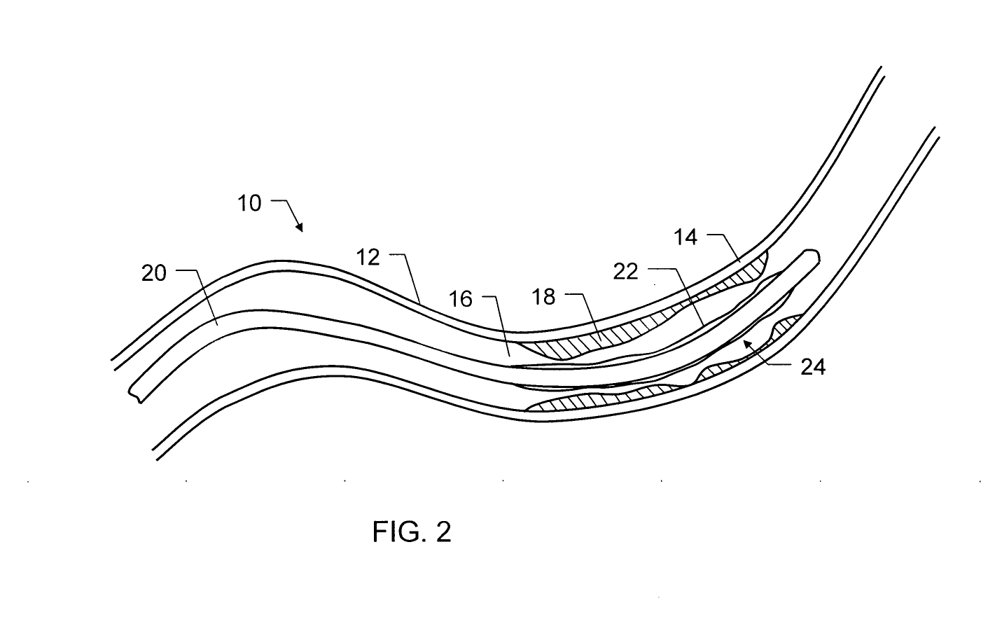

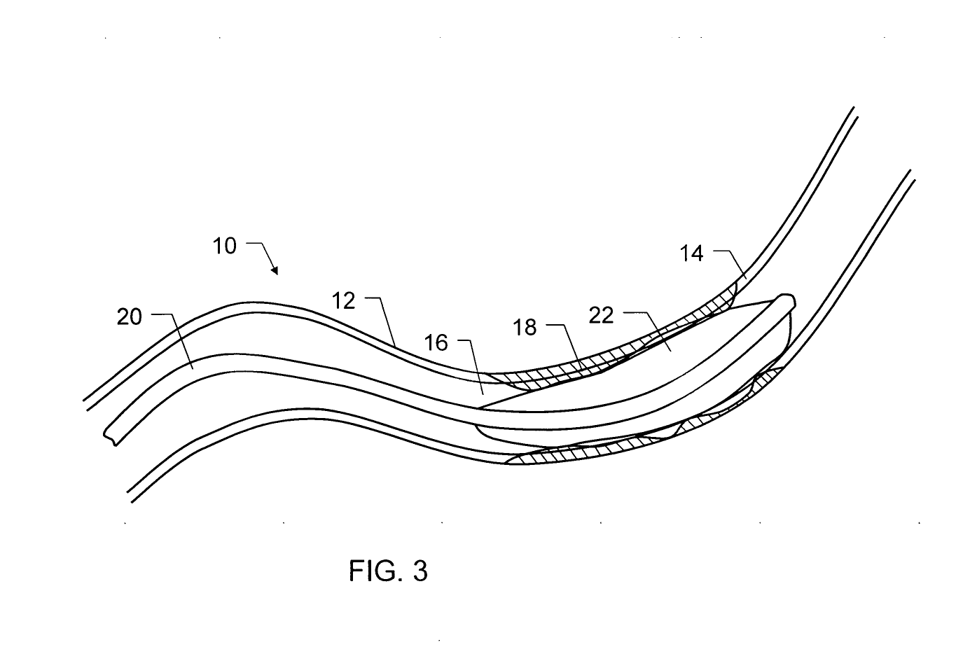

The vessel 10 may be initially treated to open up the lumen 16 at the site of the lesion 18 using known methods. For example, as illustrated in FIG. 2, a balloon catheter 20 having a balloon 22 mounted to a distal portion 24 of the catheter 20 may be tracked to the site of the lesion 18. The balloon 22 may be inflated, as shown in FIG. 3, with a fluid, as is known in the art. Inflation of the balloon 22 may push the lesion 18 radially outwardly to open the lumen 18. A stent (not shown) may also be deployed at the site of the lesion 18 to help keep the lumen 16 open, as is known in the art.

After the lumen 16 has been opened at the site of the lesion 18, there may be a risk of restenosis, as discussed above. To reduce the risk, a cuff device 30 according to embodiments of the invention may be used to treat the site of the lesion 18 with a therapeutic substance or agent. The cuff device 30 is configured to allow clinician to locate the site of the lesion 18 and apply the therapeutic substance to the site of the lesion 18, as discussed in further detail below. Depending on the location of the vessel 10, key-hole surgery or a direct cut down may be used to access the vessel 10, as known in the art, so that the cuff device 30 may be attached to the outer surface 12 of the vessel 10 at the site of the lesion 18.

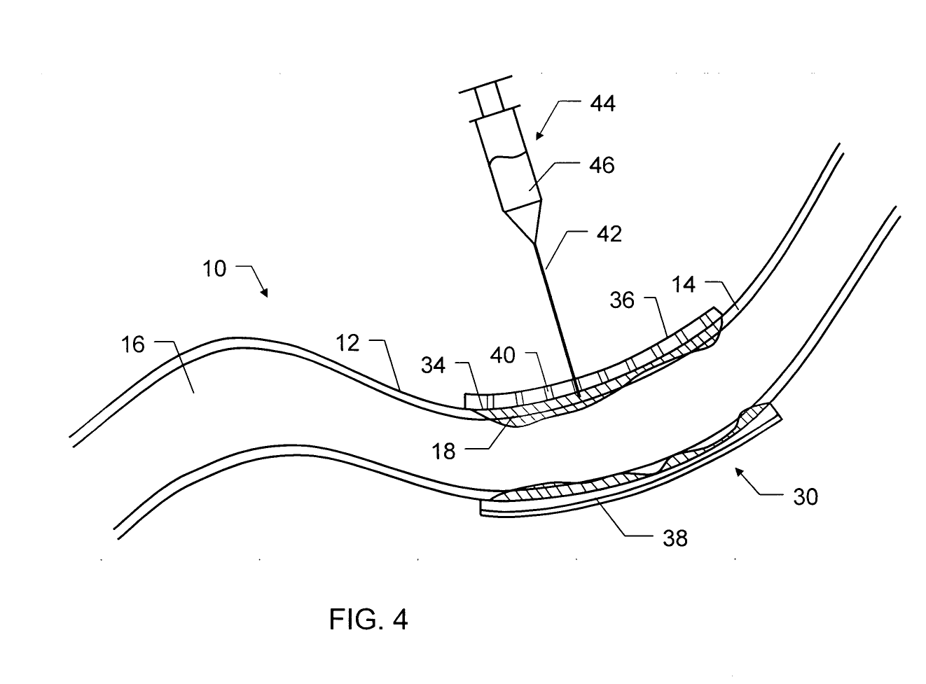

As shown in FIG. 4, the cuff device 30 may be configured to be wrapped around the outer surface 12 of the vessel 10 at the site of the lesion 18. Although the illustrated embodiment shows the cuff device 30 having a longitudinal length that is about the same as the length of the lesion 18 along the vessel 10, the cuff device 30 may be longer or shorter than the lesion 18, as desired.

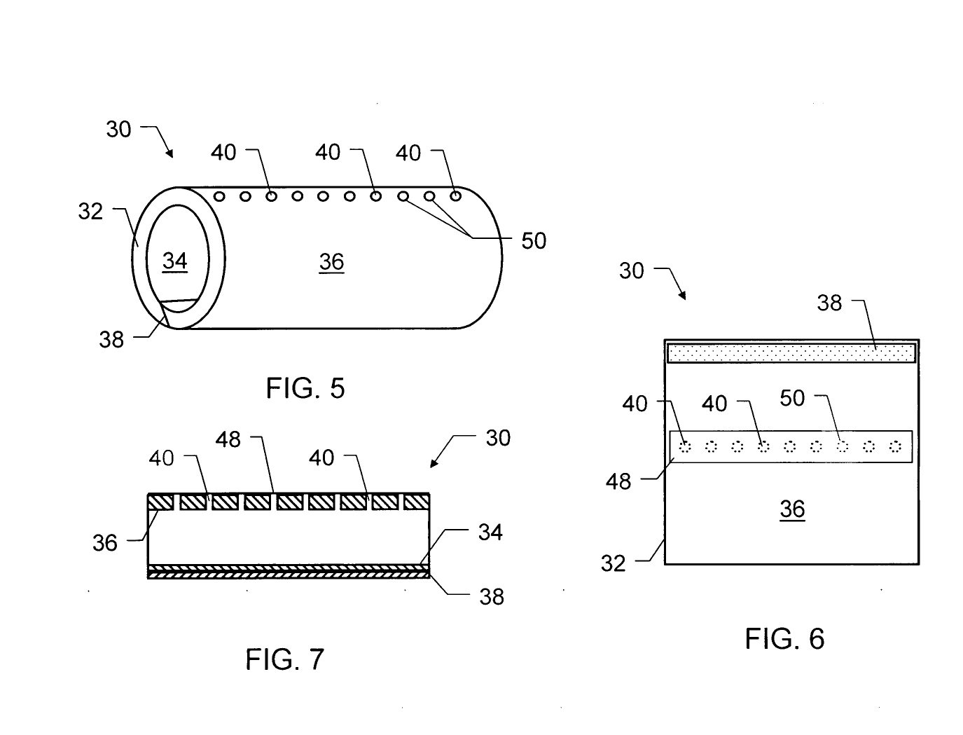

FIGS. 5-7 schematically illustrate addition details of an embodiment of the cuff device 30. As illustrated, the cuff device 30 includes a flexible material 32 having a first surface 34 and a second surface 36. The flexible material 32 may be wrapped around the vessel 10 so that the first surface 34 comes into contact with the outer surface 12 of the vessel 10, as shown in FIG. 4. The flexible material 32 may have enough flexibility to substantially conform to the outer surface 12 of the vessel 10. By conforming with the outer surface 12 of the vessel 10, any potential gaps between the flexible material 32 and the vessel 10 may be minimized.

The flexible material 32 may include a flexible mesh of fibers that may be woven together. In an embodiment, the fibers may comprise polyethylene terephthatlate (“PET”), such as PET fibers sold under the trademark DACRON. Once the cuff device 30 is wrapped around the vessel 10, it desirably flexes in the radial direction, as the vessel 10 moves, yet not move relative to the vessel 10, such as in a longitudinal direction along the vessel 10.

The flexible material 32 may be secured around the vessel 10 by use of an attachment structure 38 that is configured to allow the flexible material 32 to wrap around the vessel 10 and onto itself, similar to a self-adhering bandage that is wrapped around a finger. The attachment structure 38 may include an adhesive, a hook and loop structure, or any other suitable material or configuration that allows the cuff device 30 to be secured in place. In an embodiment, an adhesive in between the first surface 34 of the flexible material 32 and the outer surface 12 of the vessel 10 may be used in addition to the attachment structure 38 for securement of the cuff device 30 directly to the vessel 10.

As illustrated in FIGS. 4-7, the cuff device 30 also includes a plurality of openings 40, or so-called ports. Each opening 40 extends through the flexible member 32 from the first surface 34 to the second surface 36, and is configured to receive a needle 42 of a syringe 44, as shown in FIG. 4, so that the needle 42 may be inserted into the wall 14 of the vessel 10 and even into the lesion 18. The syringe 44 may be loaded with a therapeutic substance or agent 46. Examples of therapeutic substances or agents that may be used include but are not limited to an antisense agent, an antineoplastic agent, an antiproliferative agent, an antithrombogenic agent, an anticoagulant, an antiplatelet agent, an antibiotic, an anti-inflammatory agent, a therapeutic peptide, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, or combinations thereof.

As illustrated, the openings 40 are located longitudinally along the cuff device 30. When the cuff device 30 is secured to the vessel 10, the openings 40 are desirably positioned to allow easy access to the openings 40 by the needle 42. For example, when the cuff device 30 is attached to the vessel 10, the openings 40 should face outwardly and towards the skin of the patient so that the distance between the skin and the openings 40 is as short as possible. Such positioning of the openings 40 may allow for easy access by the syringe 44 and minimize any discomfort to the patient.

The use of the openings 40 in the cuff device 30 may eliminate the need to preload the above-described prosthetic device with the therapeutic substance. The use of the openings 40 may also eliminate the need to configure the prosthetic device to elute the substance at a well-defined predetermined rate. The openings 40 of the cuff device 30 may offer direct control over the type and amount of therapeutic substance that is injected, as well as the timing of the injections. Also, the openings 40 incorporate the option of providing a very specific injection location within the lesion site itself.

As illustrated in FIGS. 6 and 7, each opening 40 may include or be covered by a self-sealing membrane 48. The self-sealing membrane 48 is constructed and arranged to allow the needle 42 to be inserted into the opening 40, and reseal the opening 40 when the needle has been removed from the opening 40. The self-sealing membrane 48 may comprise of a silicone material. A self-sealing silicone membrane or septum is known in vascular access systems to provide access for infusion of fluids and/or aspiration of blood. As such, further details of the self-sealing membrane 48 are not described herein.

Although the self-sealing membrane 48 is illustrated as being a single membrane that covers all of the openings 40 on the second surface 36 of the flexible material 32, the illustrated embodiment should not be considered to be limiting in any way. For example, in an embodiment, individual self-sealing membranes may be provided to each opening at either end of the opening 40, i.e., at the first surface 34 or at the second surface 36 of the flexible material 32, or even within each opening 40.

The cuff device 30 may also include a radiopaque material in the form of a radiopaque marker 50 that may be positioned beside each opening 40, or even line each opening 40. The radiopaque markers 50 may assist the clinician with locating the openings 40 in the cuff device 30 so that the needle 42 of the syringe 44 may be properly aligned during treatment. Also, the radiopaque markers 50 may assist the clinician with the initial placement of the cuff device 30 on the vessel 10 so that the openings 40 are properly oriented, as discussed above.

The radiopaque marker 50 may be any suitable marker that is configured to be visible in a radiographic image, such as an x-ray or fluoroscopic image. Radiopaque materials do not allow certain wavelengths of radiation, such as x-rays, to pass through, which allows the clinician to see the radiopaque material in a human body when using suitable visualization equipment, such as a fluoroscope. Any suitable radiopaque material that allows the radiopaque marker 50 to be imaged with imaging equipment, while allowing the cuff device 30 to maintain flexibility, may be used. Examples of such radiopaque materials include, but are not limited to metals such as gold, platinum, and alloys thereof, and filled polymeric materials, such as barium sulfate loaded silicone, polyimide, and polycarbonate.

As shown in FIG. 4, once the cuff device 30 has been attached to the desired location on the vessel 10, a clinician may insert the needle 42 of the syringe 44 into and through an opening 40 so that the therapeutic substance 46 may be injected at the desired location of the site of the lesion 18. The same opening 40 or a different opening 40 may be subsequently used by the clinician to deliver another injection of the same or a different therapeutic substance.

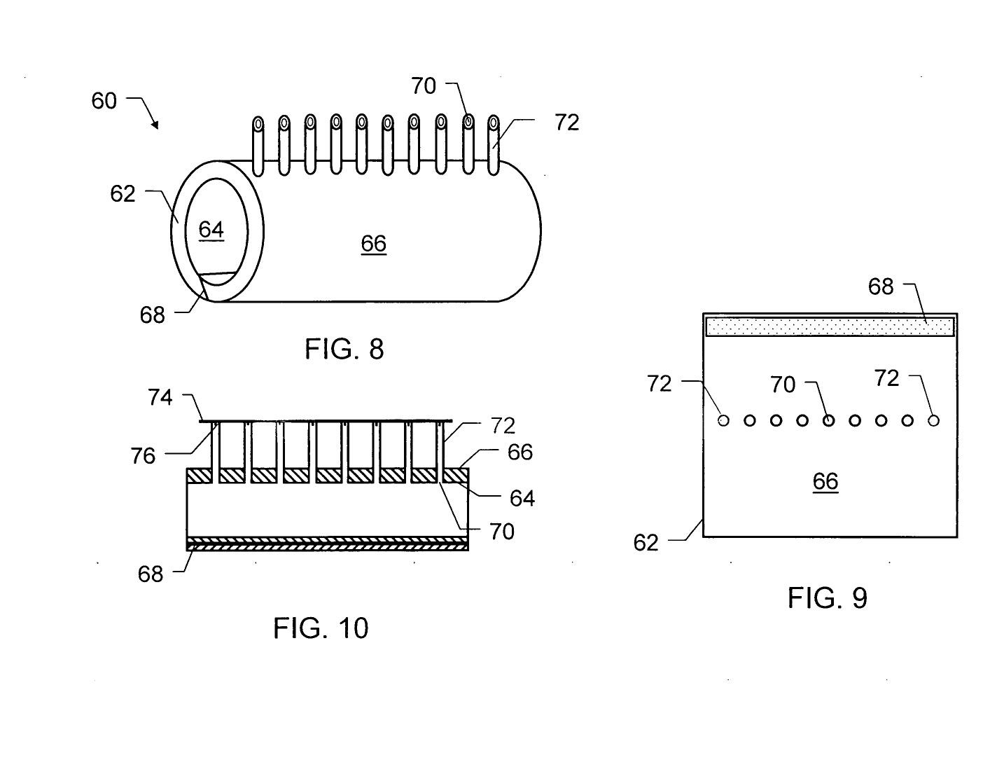

FIGS. 8-11 illustrate a cuff device 60 according to an embodiment of the invention. As illustrated, the cuff device 60 includes a flexible material 62 having a first surface 64 and a second surface 66. Similar to the flexible material 32 described above, the first surface 64 of the flexible material 62 is configured to come into contact with the outer surface 12 of the vessel 10 and to substantially conform to the outer surface 12 of the vessel 10.

The flexible material 62 may be secured around the vessel 10 with an attachment structure 68, such as an adhesive or a hook and loop structure, which is configured to allow the flexible material 62 wrap around the vessel 10 and onto itself. Similar to the flexible material 32 described above, the flexible material 62 may include a mesh of woven fibers, such as PET fibers sold under the trademark DACRON, or any other suitable material. In an embodiment, an adhesive may be used in between the first surface 64 of the flexible material 62 and the outer surface 12 of the vessel 10 for additional securement to the vessel 10. The cuff device 60 is configured to be able to flex in the radial direction with movement of the vessel 10, yet not move longitudinally along the vessel 10, once the cuff device 60 is wrapped around the vessel 10.

The cuff device 60 also includes a plurality of openings 70, or so-called ports. Similar to the openings 40 described above, each opening 70 extends through the flexible member 62 from the first surface 64 to the second surface 66. Each opening 70 may be defined by a tube 72 that extends from the first surface 64 of the flexible material 62, through the flexible material 62, and outward from the second surface 66 of the flexible material 62.

In an embodiment, the tube 72 may not pass through the flexible material 62 but may instead be attached to the second surface 66 of the flexible material 62 so that it is aligned with the opening 70. In such an embodiment, the opening 70 is not defined by the tube 72, but may have a diameter that is substantially the same as the inner diameter of the tube 72 and be aligned with the tube 72 so that the needle 42 described above may be inserted smoothly through the tube 72 and the opening 70.

Each tube 72 is configured to receive the needle 42 of the syringe 44 so that the needle 42 may be inserted into the wall 14 of the vessel 10 and even into the lesion 18, as described above. Once the needle 42 has been inserted into the desired location of the wall 14 or lesion 18, the therapeutic substance or agent 46, such as any of the substances or agents listed above, may be injected to the site of the lesion 18. Similar to the embodiment of the cuff device 30 described above, the tubes 72 and openings 70 are located longitudinally along the cuff device 60 so that the therapeutic substance 46 may be injected to the desired location along the site of the lesion 18.

The tubes 72 may be constructed and arranged to be visible in a radiographic image, such as an x-ray or fluoroscopic image, during placement of the cuff device 60 on the vessel 10. In an embodiment, each tube 72 is made from a radiopaque material, such as gold, platinum, or an alloy thereof, or a filled polymeric material, such as barium sulfate loaded silicone, polyimide, or polycarbonate, or any other suitable radiopaque material.

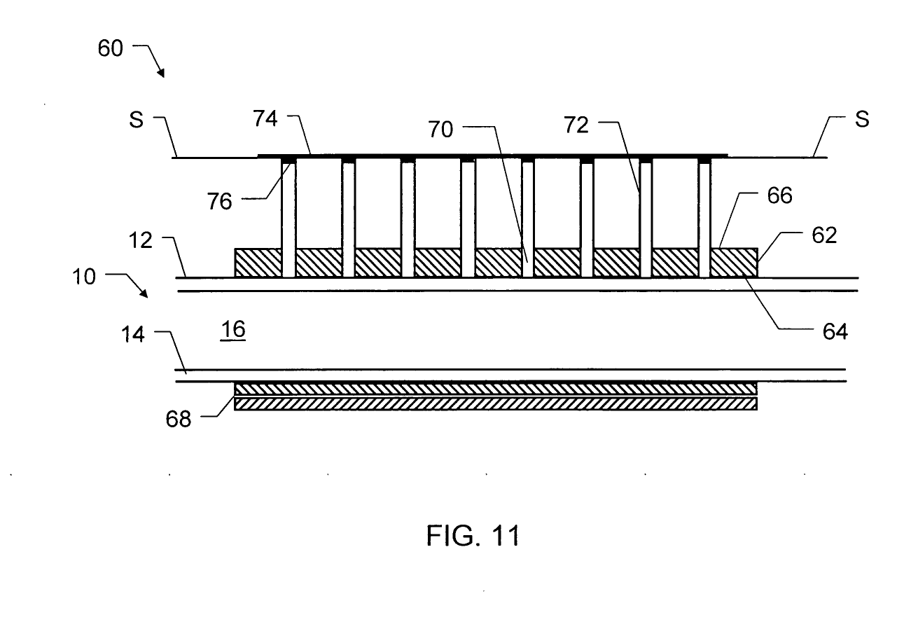

As illustrated in FIG. 11, each tube 72 may have a length that extends from the outer surface 12 of the vessel 10 being treated through the skin S of the patient. Such a configuration provides direct access to the vessel 10 without having to use fluoroscopy to locate the openings 70 in order to inject the therapeutic substance 46. By locating the end of the tube 72 at the patient's skin S, the clinician may pass the needle 42 of the syringe 44 directly into the tube 72 and opening 70 of the flexible membrane 62, without having to locate the opening 70 or pierce the patient's skin S with the needle 42.

As shown in FIGS. 10 and 11, the cuff device 60 may also include a cover 74 that is constructed and arranged to cover the tubes 72. The cover 74 may also be configured to cover a portion of the skin S in the vicinity of the tubes 72. The cover 74 is configured to externally close off the tubes 72, and therefore the cuff device 60, so that foreign matter may not unintentionally enter the cuff device 60. Also, the cover 74 may prevent substances from exiting the patient's body through the tube 72. As illustrated, the cover 74 may include a plurality of projections 76. Each projection 76 may be configured to be inserted into a corresponding tube 72 and engage an inner wall of the tube 72 to help secure the cover 74 to the tubes 72. A bandage (not shown) may also be used to cover the tubes 72 and/or the cover 74.

Although not depicted in FIGS. 8-11, the cuff device 60 may also include a self-sealing membrane similar to the self-sealing membrane 48 described above. The self-sealing membrane may be used in addition to or in place of the cover 74. The illustrated embodiment is not intended to be limiting in any way.

Embodiments of the cuff device 30, 60 described herein may be particularly suitable to treat chronic lesions, especially lesions located in vessels in the lower limbs, such as the femoral artery, the peroneal artery, and posterior tibial artery. The cuff device 30, 60 may be surgically attached to the vessel 10 and configured to be in place for an extended period of time. Continuous treatment of the affected site with suitable therapeutic substances may be provided by using the plurality of openings 40, 70 in the flexible member 32, 62. Such continuous treatment may assist in the prevention of restenosis, especially for high risk patients, including but not limited to diabetics.

If desired, the cuff device 30, 60 may be removed by surgically accessing the vessel 10 when treatment has concluded, or if it is desirable to replace the cuff device 30, 60. A new cuff device 30, 60 having the same or a different configuration may be wrapped around the vessel 10 so that treatment may continue, if desired.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient roadmap for implementing an exemplary embodiment of the invention, it being understood that various changes may be made in the function and arrangement of elements described in an exemplary embodiment without departing from the scope of the invention as set forth in the appended claims.

What is claimed is:

1. A cuff device configured to treat a site of a lesion in a vessel, the cuff device comprising:
a flexible material having a first surface and a second surface, the flexible material being configured to be wrapped around an exterior surface of the vessel so that the first surface of the flexible material is in contact with an exterior surface of the vessel; and
an opening that extends from the first surface to the second surface, the opening being constructed and arranged to allow a needle of a syringe to pass therethrough.

2. A cuff device according to claim 1, further comprising:
a self-sealing membrane that covers the opening, the self-sealing membrane being constructed and arranged to allow the needle to pass through the membrane so that the needle may pass through the cuff device and into the vessel when the flexible material is wrapped around the exterior surface of the vessel.

3. A cuff device according to claim 2, wherein the self-sealing membrane comprises silicone.

4. A cuff device according to claim 1, further comprising a plurality of openings that extend from the first surface to the second surface of the flexible material, the self-seal member being configured to cover the plurality of openings.

5. A cuff device according to claim 1, wherein the flexible material comprises a woven mesh of fibers.

6. A cuff device according to claim 5, wherein the fibers comprise polyethylene terephthalate.

7. A cuff device according to claim 1, further comprising a radiopaque material located adjacent the opening.

8. A cuff device according to claim 1, wherein the opening is lined with a radiopaque material.

9. A cuff device according to claim 1, further comprising a tube extending from the second surface of the flexible material at the opening, the tube being constructed and arranged to receive the needle.

10. A cuff device according to claim 9, wherein the tube comprises a radiopaque material.

11. A cuff device according to claim 10, further comprising a cover constructed and arranged to cover the tube.

12. A cuff device according to claim 1, further comprising an adhesive to secure the flexible material to the vessel.

13. A cuff device according to claim 1, further comprising an attachment structure configured to attach one portion of the flexible material to another portion of the flexible material to secure the flexible material to the vessel.

14. A method for treating a site of a lesion in a vessel, the method comprising:
placing a flexible material around an exterior surface of the vessel;
locating an opening in the flexible material;
passing a needle of a syringe through the opening and into the vessel; and
injecting the vessel with a therapeutic substance with the syringe.

15. A method according to claim 14, further comprising securing the flexible material to the vessel.

16. A method according to claim 14, further comprising retracting the needle out of the opening and sealing the opening.

17. A method according to claim 14, further comprising radially expanding the vessel at the lesion with a balloon catheter prior to placing the flexible material around the exterior surface of the vessel.

18. A method for treating a site of a lesion in a vessel, the method comprising:
placing a flexible material of a cuff device around an exterior surface of the vessel;
passing a tube of the cuff device that extends from the flexible material through skin;
passing a needle of a syringe through the tube and through an opening in the flexible material; and
injecting the vessel with a therapeutic substance with the syringe.

19. A method according to claim 18, further comprising securing the flexible material to the vessel.

20. A method according to claim 18, further comprising covering the tube and a portion of the skin with a cover.

21. A method according to claim 18, further comprising radially expanding the vessel at the lesion with a balloon catheter prior to placing the flexible material of the cuff device around the exterior surface of the vessel.
